(19) United States
(12) Patent Application Publication
(10) Pub. No.: US 2015/0173688 A1
(43) Pub. Date: Jun. 25, 2015

(54) MEDICAL MONITOR

(71) Applicant: Derak Joseph RANKE, East Dublin, GA (US)

(72) Inventor: Derak Joseph RANKE, East Dublin, GA (US)

(21) Appl. No.: 14/139,329

(22) Filed: Dec. 23, 2013

(51) Int. Cl. A61B 5/00; A61B 5/02; G08B 25/01

(52) U.S. Cl. CPC A61B 5/746; G08B 25/016; A61B 5/02042; A61B 5/6802

(57) ABSTRACT

A medical monitor is provided. The medical monitor may include a plurality of sheets and a power source electrically connected in series with an alarm and an open circuit. The plurality of sheets may include a distal side and a proximal side. The open circuit may be sandwiched between distal sides of adjacent sheets. A user may mount either proximal side to a bleed site on a patient where bleeding can occur, for example, entry points for intravenous applications. The plurality of sheets may allow blood to flow from the bleed site through the proximal side and through distal side so that the blood may close the open circuit, so that the alarm is powered.

### BACKGROUND OF THE INVENTION

The present invention relates to medical monitoring systems and, more particularly, to alerting caregivers when a patient is bleeding.

Many devices have been proposed to detect when patients may be bleeding. Currently, such devices are expensive considering the dictates of the medical field that demand disposal of blood contaminated items after one use. Moreover, current devices involve many and sometimes bulky parts the preclude adapting the device effectively to problematic and otherwise hard to reach bleed sites.

As can be seen, there is a need for a medical monitoring system that can alert caregivers when a patient is bleeding and that is also adaptable for problematic and otherwise hard to reach bleed sites. Moreover, it is desirable and advantageous that the medical monitoring system be inexpensive given the mandated disposal and repurchase of the medical monitoring system after one use.

### SUMMARY OF THE INVENTION

In one aspect of the present invention, a medical monitor comprises: an alarm; a power supply; an open circuit comprising two spaced electrically conductive elements electrically connected in series with the alarm and the power supply; and a first sheet comprising a distal side and a proximal side, wherein the distal side is connected to the open circuit.

In another aspect of the present invention, a method of detecting blood comprises: providing a medical monitor comprising: an alarm; a power supply; an open circuit comprising two spaced electrically conductive elements electrically connected in series with the alarm and the power supply; and two sheets comprising a distal side and a proximal side, wherein the distal sides sandwich the open circuit; and mounting the two sheets over a bleed site.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention;

FIG. 2 is a plan view of an exemplary embodiment of the alarm circuitry of the present invention; and

FIG. 3 is a plan view of an exemplary embodiment of the open circuit of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a medical monitor. The medical monitor may include a plurality of sheets and a power source electrically connected in series with an alarm and an open circuit. The plurality of sheets may include a distal side and a proximal side. The open circuit may be sandwiched between distal sides of adjacent sheets. A user may mount either proximal side to a bleed site on a patient where bleeding can occur, for example, entry points for intravenous applications. The plurality of sheets may allow blood to flow from the bleed site through the proximal side and through the distal side so that the blood may close the open circuit, so that the alarm is powered.

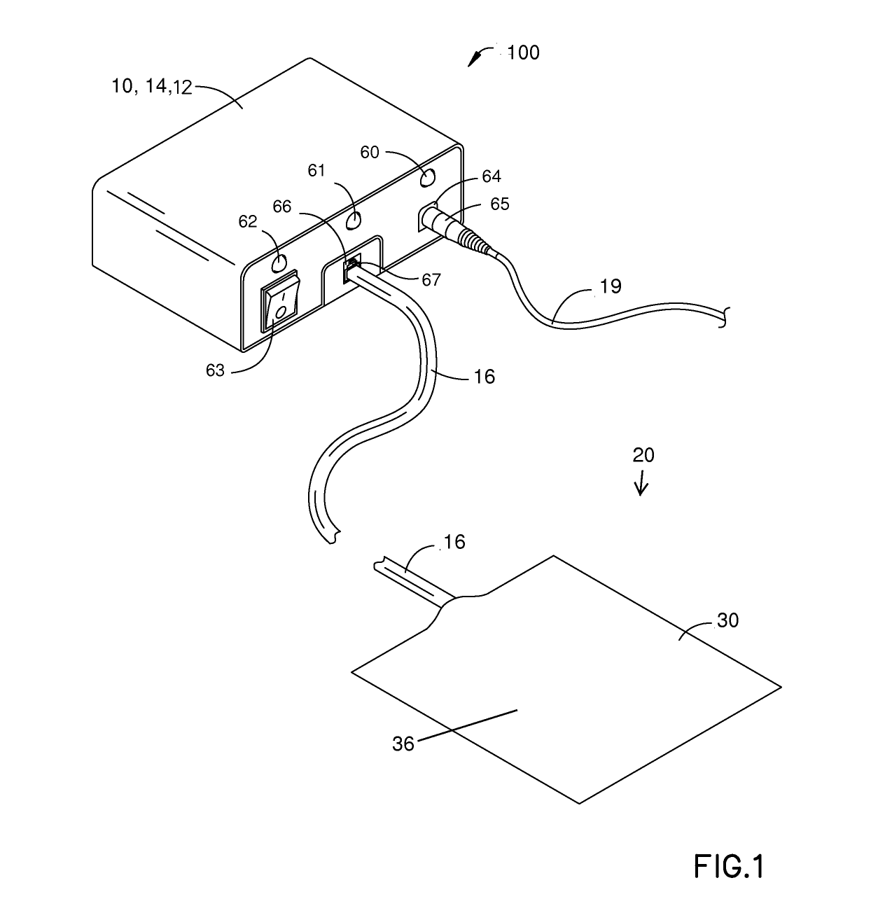

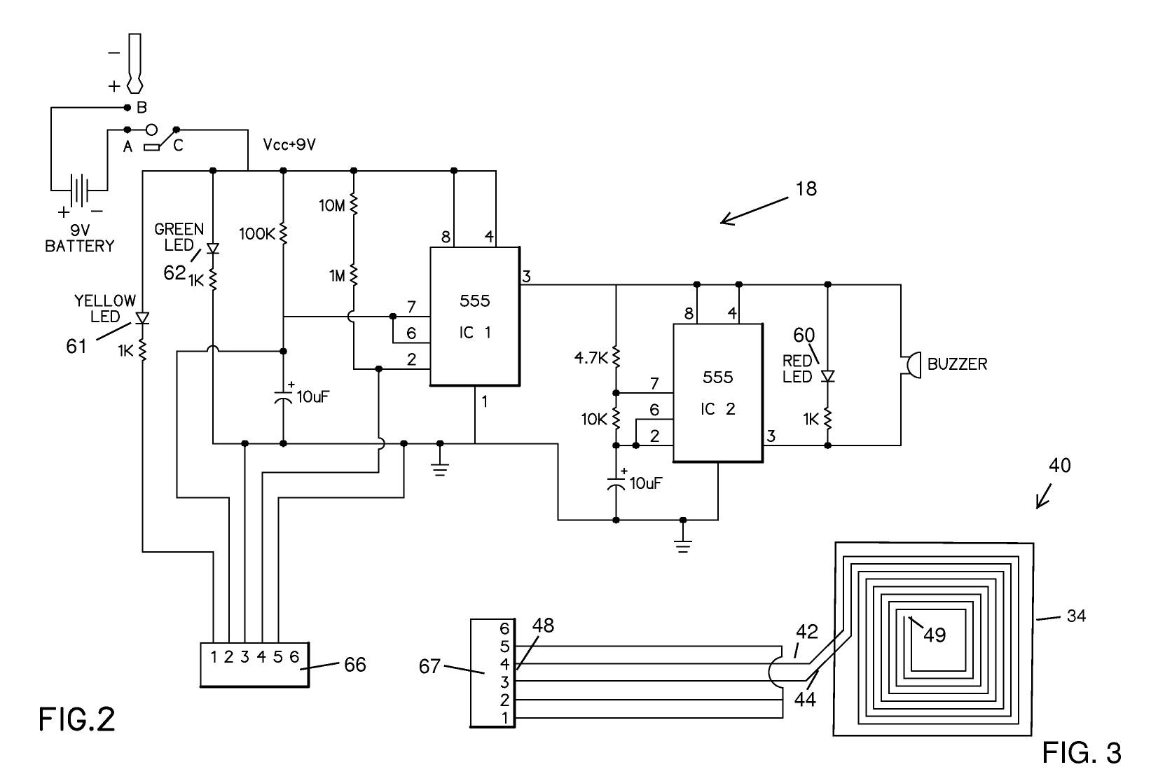

Referring to FIGS. 1 through 3, the present invention may include a medical monitor 100. The medical monitor 100 may include a power alarm 10 and a blood sensor 20.

The blood sensor 20 may include a plurality of sheets 30 sandwiching an open circuit 40. The plurality of sheets 30 may include porous, non-conductive material. The plurality of sheets 30 may include a distal side 34 and a proximal side 36. The proximal side 36 may be adapted for mounting directly on a patient. The distal side 34 may be adapted for encasing the open circuit 40.

The plurality of sheets 30 material may include gauze, bandage, fabric with a loose open weave, netting type structure and the like that is sufficiently pliable to adapt the blood sensor 20 to problematic and hard to reach bleed sites. The plurality of sheets 30 thickness and material may be sufficiently permeable so as to allow free flow of blood from and through the proximal side 36 to and through the distal side 34. The plurality of sheets 30 thickness and material may be sufficiently impermeable so as to prevent the free flow of non-blood fluid from and through the proximal side 36 to and through the distal side 34. The plurality of sheets 30 may have various shapes, such as, square, rectangular, circular, oval, trapezoidal, triangular, u-shape, etc.

In certain embodiments, the plurality of sheets 30 may be a collection of separate sheets 30 stacked together, wherein the distal side 34 of one sheet 30 removably attached to the proximal side 36 of an adjacent sheet 30. The removable attachment between adjacent sheets 30 may be the result of friction forces, adhesive substances, combinations thereof, and the like

The open circuit 40 may be held and supported by at least one distal side 34. The open circuit 40 may include a first element 42 and a second element 44. The first element 42 and the second element 44 may be electrical wire, electrical conductors and the like, that may be sufficiently malleable to adapt the blood sensor 20 to problematic and hard to reach bleed sites. The first element 42 and the second element 44 may run parallel and be spaced apart at a predetermined distance. The open circuit 40 may conform to the shape of the plurality of sheets 30 so that a portion of the open circuit 40 may be present over substantially all of the surface area of the distal side 34. The open circuit 40 may terminate at a port end 48 and at a sensor end 49. The port end 48 may include a plug 67.

In certain embodiments, the sensor end 49 may terminate near an opening disposed within the plurality of sheets 30. The opening may be mounted around the bleed site.

The power alarm 10 may include an alarm 12, a voltage source 14, an alarm circuitry 18, a cord 16, a jack 66 and a power source 19 for powering the power alarm 10. The alarm 12 may be of various types such as, but not limited to, noise-making, light-activating, vibration-emitting types and the like, sufficient to alert all necessary attendants.

In certain embodiments, the power alarm 10 may include an alarm light 60, a notification light 61, a power light 62, a power switch 63 and a power input port 64. The power source 19 may include a power jack 65 connectable to the power input port 64.

The cord 16 may electrically connect the blood sensor 20 to the power alarm 10. In certain embodiments, the cord 16 may terminate in the plug 67 connectable to the jack 66. The voltage source 14 may apply a potential difference between the first element 42 and the second element 44.

The alarm circuitry 18 may be configured so that when the first element 42 and the second element 44 circuit may be bridged by blood the alarm 12 receives power. The alarm circuitry 18 may be configured for dialysis settings. In an alternative embodiment, the power source 19 may be the voltage source 14 electrically connected in series with the alarm 12 and the open circuit 40 so that blood may close the circuit so as to power the alarm 12.

A method of using the present invention may include the following. The medical monitor 100 disclosed above may be provided. A user may mount either proximal side 36 of the plurality of sheets 30 to a bleed site on a patient where bleeding can occur, for example, entry points for intravenous applications. The plurality of sheets 30 may allow blood to flow from the bleed site through the proximal side 36 and to and through the distal side 34 so that the blood may contact the open circuit 40. If sufficient bleeding occurs, the blood may bridge the predetermined distance between the first element 42 and the second element 44 so as to close the open circuit 40.

In certain embodiments, the user may add additional layer of the sheets 30 by mounting one sheet's 30 proximal side 36 to an adjacent sheet's 30 distal side 34. The additional layers may be used as an intermediate barrier between the bleed site and the open circuit 40 to adjust the sufficient of blood needed to close the open circuit 40.

In certain embodiments, the plurality of sheets 30 may be tailored in shape and size to accommodate various sizes and locations of bleed sites. In certain embodiments, the medical monitor 100 may be placed between the part of the patient's body part containing the bleed site and where that body part rests. In an alternate embodiment, the medical monitor 100 may be placed on the floor or bed where the patient is at rest.

From the description above, a number of advantages of some of the embodiments become evident. For example, the plurality of sheets 30 can be made from cost effective material, whereby a few rolls of it will permit the manufacturer to cost effectively mass produce the medical monitor 100. This would be a boon in the medical field where blood-contaminated devices are required to be thrown away after one use. Likewise, the medical monitor 100 is pliable and of adaptable construction, allowing a user to apply the present invention to problematic and hard to reach bleed sites. Moreover, either side of the blood sensor 20 may detect blood and may be mounted to the patient.

It should be understood, of course, that the foregoing relates to exemplary embodiments of the invention and that modifications may be made without departing from the spirit and scope of the invention as set forth in the following claims.

What is claimed is:

1. A medical monitor comprising:
an alarm;
a power supply;
an open circuit comprising two spaced electrically conductive elements electrically connected in series with the alarm and the power supply; and
at least a first sheet comprising a distal side and a proximal side, wherein the distal side is connected to the open circuit.

2. The medical monitor of claim 1, further comprising a second sheet comprising a distal side and a proximal side, wherein the distal sides of the first sheet and the second sheet releasably connect and sandwich the open circuit forming a blood sensor.

3. The medical monitor of claim 2, further comprising at least one adjacent sheet comprising a distal side and a proximal side, wherein the distal side is configured to releasably connect to the proximal side of the first sheet, the second sheet, and a separate adjacent sheet.

4. The medical monitor of claim 2, wherein the first sheet, the second sheet, and the open circuit each comprise an opening, wherein each opening align forming the opening in the blood sensor.

5. A medical monitor comprising:
an alarm;
a voltage source operatively connected to the alarm;
an open circuit comprising two spaced electrically conductive elements electrically connected to the voltage source so that the voltage source applies a potential difference between the two spaced electrically conductive elements;
an alarm circuitry operatively connecting the open circuit and the alarm, wherein the alarm circuitry is configured to activate the alarm when the two spaced electrically conductive elements are bridged by blood; and
at least a first sheet comprising a distal side and a proximal side, wherein the distal side is connected to the open circuit.

6. The medical monitor of claim 5, further comprising a second sheet comprising a distal side and a proximal side, wherein the distal sides of the first sheet and the second sheet releasably connect and sandwich the open circuit forming a blood sensor.

7. The medical monitor of claim 6, further comprising at least one adjacent sheet comprising a distal side and a proximal side, wherein the distal side is configured to releasably connect to the proximal side of the first sheet, the second sheet, and a separate adjacent sheet.

8. The medical monitor of claim 6, wherein the first sheet, the second sheet, and the open circuit form substantially identical openings so that the blood sensor forms the substantially identical opening.

9. The method of detecting blood comprising:
providing a medical monitor comprising: an alarm; a power supply; an open circuit comprising two spaced electrically conductive elements electrically connected in series with the alarm and the power supply; and two sheets comprising a distal side and a proximal side, wherein the distal sides sandwich the open circuit; and
mounting the two sheets over a bleed site.

10. The method of claim 9, wherein the two sheets are mounted under the bleed site.
